(19) United States
(12) Patent Application Publication
(10) Pub. No.: US 2024/0207215 A1
(43) Pub. Date: Jun. 27, 2024

(54) MARC1 INHIBITORS FOR TREATMENT OF LIPID METABOLISM DISORDERS

(71) Applicants: Bernd Clement, Altenholz (DE); Thomas Kunze, Scharnhagen (DE); Patrick Marcel Indorf, Ganderkesee (DE); Sophia Rixen, Kiel (DE)

(72) Inventors: Bernd Clement, Altenholz (DE); Thomas Kunze, Scharnhagen (DE); Patrick Marcel Indorf, Ganderkesee (DE); Sophia Rixen, Kiel (DE)

(21) Appl. No.: 18/010,045

(22) PCT Filed: Jun. 17, 2021
(86) PCT No.: PCT/EP2021/066416

§ 371 Date: Feb. 13, 2023

(30) Foreign Application Priority Data
Jun. 17, 2020 (DE) 10 2020 003 608.2

(51) Int. Cl. A61K 31/27

(52) U.S. Cl. CPC A61K 31/27

(57) ABSTRACT

A method for the prevention and treatment of obesity, non-alcoholic fatty liver disease, alcoholic fatty liver disease, non-alcoholic steatohepatitis, liver cirrhosis, liver fibrosis, elevated liver enzyme levels (ALT, AST, ALP), hepatocellular carcinoma, hypercholesterolemia and cardiovascular diseases associated therewith, insulin resistance, impaired glucose tolerance, hyperglycemia, type II diabetes mellitus, and metabolic syndrome. This method involves the inhibition of mARC1 in the patient by chemical compounds (retardants, inhibitors) of the general structure:

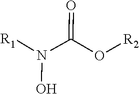

Inhibitors and retardants refer to all chemical compounds that reduce or downregulate the conversion rate of mARC1. Downregulation in the patient means that a lower conversion rate of reactions catalyzed by mARC1 is achieved in the patient by the inhibitors in a drug. Similarly, protein-protein interactions involving mARC1 are downregulated in the patient by a drug with the described compounds. Moreover, inhibitors also include all substances that inhibit the degradation of another drug by mARC1 so that it is present in sufficiently high therapeutic concentration.

The invention relates to compounds that are inhibitors of human mARC1 for use in the treatment of metabolic diseases, in particular lipid metabolism diseases, e.g., of obesity, non-alcoholic fatty liver diseases, alcoholic fatty liver diseases, non-alcoholic steatohepatites, liver cirrhoses, liver fibroses, elevated liver enzyme levels (e.g., elevated levels of ALT, AST, ALP), hepatocellular carcinomas, hypercholesterolemiae, and associated cardiovascular diseases, insulin resistances, impaired glucose tolerances, hyperglycemiae, type II diabetes mellitus, and metabolic syndrome.

### STATE OF THE ART

The mitochondrial amidoxime reducing component (mARC) was first described in 2006 (1). The human mARC subdivides into two paralogous forms, mARC1 and mARC2, which are encoded by the two genes MARC1 and MARC2, oriented in tandem with 5044 base pairs distance on chromosome 1. Compared to the other human molybdenum-containing enzymes such as xanthine oxidoreductase, sulfite oxidase, and aldehyde oxidase, it has a simple structure, due to it possessing only one Moco- binding domain, according to current research. Both mARC enzymes are part of a three-component system consisting of mARC, cytochrome b5, and cytochrome b5 reductase. Together, these are capable of reducing a variety of different N-oxygenated compounds under consumption of NADH (2). In this context, the reduction of hydroxamic acids as well as sulfhydroxamic acids (3), N-oxides (4), oximes (5), hydroxyurea derivatives (5) and hydroxylamines (5) has been demonstrated. Thus, mARC is involved in the metabolism of some established drugs such as hydroxyurea (5). mARC is highly conserved and has so far been found in all mammalian genomes studied. The physiological function of mARC is not yet fully elucidated, it has been shown to be involved in NO homeostasis (6, 7), lipid metabolism (8, 9) as well as the detoxification of mutagenic substances such as N-hydroxylated base analogues (10). In the human cell, mARC is localized at the outer mitochondrial membrane (1, 2). In human cells, mARC1 is predominantly present, which has the highest expression levels in adipocytes. However, mARC enzymes could be found in almost all tissues. High levels are also present in thyroid, liver, kidney and small intestine (2).

It could be shown that an absence or a reduced activity of mARC1 in humans is associated with a reduced incidence of nonalcoholic as well as alcoholic fatty liver cirrhosis subtypes. Missense variants of MARC1 are directly associated with a lower risk of overall cirrhosis. It was also shown that in the case of an absence or a decreased activity of mARC1, both reduced liver fat and blood cholesterol were present, reflected by decreased levels of low-density protein and high-density protein. In addition, the blood laboratory parameters aspartate aminotransferase and alanine aminotransferase, which are used to diagnose liver damage, were significantly decreased (11).

An association of mARC with diabetes mellitus has been known since 2007. Malik et al. showed changes in the expression of mARC2 in diabetic animal models. Among others, an increased expression compared to control of mARC2 could be detected in the kidneys of diabetic Goto Kakizaki rats (model for type 2 diabetes) (12).

In 2010, this association was corroborated by Chen et al. identifying a genetic variation for mARC2 that is associated with an increased risk of type 1 and type 2 diabetes (13).

In a mARC2 knockout mouse model, it could be shown that the absence of mARC2 is associated with a decreased body fat percentage, reduced levels of cholesterol and of triglyceride. Despite high-caloric diets (60% of calories from fat) over a 23-week period, the KO mice showed no signs of obesity. The body weight gain corresponded approximately to the body weight difference gained by the control mice on a normal diet (10% of calories from fat). Thus, a resistance of these animals to high-calorie-induced obesity was shown. To relate biochemical data to the liver status, liver histopathology was performed in each experimental group, revealing microvesicular steatosis and hepatocellular ballooning in wild-type mice on high-caloric diet, while no pathological change was present in KO animals (14).

To date, it is unclear to what extent the murine paralogs mARC1 and mARC2 correspond to the human ones in their physiological function. However, on a nucleotide sequence basis, it is clear that murine mARC1 is 80% identical to human, and for mARC2 it is 74% (15).

For the first time, life expectancy in the United States has declined. This can be attributed in large part to obesity-related “metabolic diseases” such as diabetes, kidney disease, stroke, and cardiovascular disease (16). As the number of people with diabetes and obesity increases, so does the prevalence of nonalcoholic fatty liver disease, which affects more than a quarter of adults worldwide (17) and 60% of diabetics (18). Among the obese, the prevalence rises as high as 90% (19). It is predicted that the prevalence in the U.S. will be 33.5% of the adult population in 2030 (20).

The epidemiologic shift from infectious diseases to metabolic diseases as a cause of mortality is also reflected in the changing epidemiology of liver diseases. Vaccines and therapies today prevent or cure many forms of viral hepatitis. Now it is the nonalcoholic fatty liver diseases that represent a heavy economic burden (21-23). Patients with a thereby caused terminal or worsened liver disease now represent the major group of liver transplant patients, thereby overtaking hepatitis C patients (24, 25).

Nonalcoholic fatty liver disease is defined as the presence of fat accumulation in the liver. It is associated with obesity and includes a range of pathologies such as steatosis, nonalcoholic steatohepatitis, fibrosis, cirrhosis, and even hepatocellular carcinoma (26). For the diagnosis, other causes that can trigger steatosis must be excluded. Other causes would e.g. be excessive alcohol consumption, viral hepatitis, chronic liver diseases such as Wilson's disease, hemochromatosis, viral hepatitis, autoimmune hepatitis, cholestatic liver disease, starvation, lipodystrophy, celiac disease, Cushing's disease, and medications (corticosteroids, methotrexate, diltiazem, oxaliplatin, amiodarone, isoniazid, highly active antiretroviral therapy, etc.).

The manifestation of nonalcoholic steatohepatitis is due to multiple mechanisms, including metabolic and genetic mechanisms, gut microbial factors, and environmental factors (27). It is characterized by a proinflammatory milieu that provokes cellular injury in the liver. The inability to suppress damaging processes such as oxidative stress, dysregulation of the unfolded protein response (which leads to stress in the endoplasmic reticulum), lipotoxicity, and apoptotic pathways then leads to hepatocellular injury and a progressive fibrosis. In 20% of patients who developed steatohepatitis, this results in liver cirrhosis (28). The most common cause of death in patients with liver cirrhosis is hepatocellular carcinoma that develops therefrom (26, 27, 29). Hepatocellular carcinoma is the fifth leading cause of cancer and the third leading cause of cancer-related death worldwide. Beside hepatitis C infection, non-alcoholic fatty liver disease is increasingly gaining importance as a cause (30).

The liver represents a key role in glucose and lipid metabolism, which is why it is not surprising that nonalcoholic fatty liver disease is a risk and a concomitant of many metabolic diseases. These include, for example, cardiovascular disease, type II diabetes and metabolic syndrome (31, 32).

Lipids circulating in the blood in the form of low-density proteins (LDL) and very-low-density (VLDL), as well as ectopic lipid accumulation in liver and skeletal muscle, cause insulin action in these tissues to be impeded and insulin resistance to develop (33-37). A subsequent adipocyte dysfunction promotes macrophage infiltration and increases lipolysis, whereby hepatic carbohydrate and lipid metabolism are further impeded in several ways. Increased fatty acid esterification and hepatic triglyceride synthesis occur, exacerbating hepatic steatosis, hepatic insulin resistance, and hypertriglyceridemia (35). Conversely, however, the presence of diabetes mellitus can evidently increase the risk of liver disease (38).

By reducing circulating LDL, (and increasing high-density lipoproteins (HDL)); the therapeutic benefit extends to a reduction in the risk of cardiovascular disease (39). Nonalcoholic fatty liver disease is evidently directly related to cardiovascular disease (40). According to statements of the World Health Organization, cardiovascular disease is the leading cause of death worldwide, responsible for an estimated 18 million deaths per year (41).

Hyperlipidemia and hyperglycemia are major medical conditions which are summarized in the metabolic syndrome, thus nonalcoholic fatty liver disease is also associated with the metabolic syndrome (42). A metabolic syndrome is present when at least three of the following features are present in a patient: elevated blood pressure, elevated fasting blood glucose, elevated triglyceride levels, elevated LDL and decreased HDL levels in the blood, a waist circumference in men >102 cm in women >88 cm. Approximately 90% of patients with non-alcoholic fatty liver disease have at least one of these characteristics, approximately 33% meet the criteria for a diagnosis of ‘metabolic syndrome’ (43).

The liver also plays a central role in the metabolism of alcohol. Upon excessive alcohol consumption, a pathophysiological alteration of metabolic processes occurs, such as e.g. decreased degradation of acetyl-CoA. Excess acetyl-CA leads to an increase in fatty acid synthesis. As a result, about 90% of all alcoholics suffer from fatty liver (44-46).

The listed potential consequences of nonalcoholic fatty liver disease, as well as its increasing prevalence, make the identification of strategies to reduce liver lipids critical.

Currently, exercise and caloric restriction are the only approved available treatment options for nonalcoholic fatty liver disease (47). However, these interventions, although effective when followed, are met with poor compliance (48, 49). Given the lack of pharmaceutical options, intensive research has been conducted to identify new compounds.

In US 2019/0160154 A1, knock-down methods for downregulation of mARC1 and mARC2 are described that act at the DNA and RNA levels.

### OBJECT OF THE INVENTION

The invention has the object of providing alternative active compounds for the treatment of metabolic diseases, in particular diseases of lipid metabolism.

### DESCRIPTION OF THE INVENTION

The invention achieves the object by the features of the claims and in particular by active compounds which are inhibitors of human mARC1 for use in the treatment and prevention of diseases of lipid metabolism, in particular of obesity, non-alcoholic fatty liver diseases, alcoholic fatty liver diseases, non-alcoholic steatohepatitides, liver cirrhoses, liver fibroses, elevated liver enzyme levels (ALT, AST, ALP), hepatocellular carcinomas, hypercholesterolemiae and cardiovascular diseases associated therewith, insulin resistances, impaired glucose tolerances, hyperglycemiae, type II diabetes mellitus, and the metabolic syndrome. It has been shown that the inhibition of mARC as a target enables the treatment and prevention of lipid metabolism disorders.

Accordingly, the invention relates to the use of inhibitors of human mARC1 for the treatment of obesity, non-alcoholic fatty liver diseases, alcoholic fatty liver diseases, non-alcoholic steatohepatitides, liver cirrhoses, liver fibroses, elevated liver enzyme levels (ALT, AST, ALP), hepatocellular carcinomas, hypercholesterolemiae and cardiovascular diseases associated therewith, insulin resistances, impaired glucose tolerances, hyperglycemiae, type II diabetes mellitus, the metabolic syndrome, and/or a disease resulting from any of the aforementioned characteristics. The patient treated with the inhibitor can be a human or another animal.

Obesity or adiposity refers to overweight, especially severe overweight. An obese person has a body mass index (BMI) of 30 or higher.

Non-alcoholic fatty liver (steatosis) refers to a storage of lipids (mainly triglycerides) in the hepatocytes with a fat content of more than 5% of the liver weight, which is not essentially caused by increased alcohol consumption (women: ≤20 g/d, men≤30 g/d). Histologically, microvesicular (small-droplet) steatosis is distinguished from macrovesicular (large-droplet) steatosis. Underlying causes of this pathological change can i.a. be (abdominal, visceral) obesity, type II diabetes mellitus and hyperlipidemia—subcomponents of the so-called metabolic syndrome—; drugs or toxins and (rarely) inborn metabolic disorders (e.g., A-/hypobetalipoproteinemia) or hormonal imbalances, as in polycystic ovary syndrome.

Alcoholic fatty liver refers to steatosis with excessive alcohol consumption as the underlying cause (women: >20 g/d, men>30 g/d).

Non-alcoholic/alcoholic steatohepatitis refers to the condition when, in addition to steatosis, mixed cell inflammatory infiltrates are detectable in the hepatic lobules and hepatocellular damage in the form of ballooning, necroapoptosis with or without fibrosis is also present. It is considered a progressive subtype of non-alcoholic/alcoholic fatty liver

Liver fibrosis refers to the condition of excessive accumulation of extracellular matrix proteins, mainly collagen, that occurs in most types of chronic liver disease. An advanced liver fibrosis leads to liver cirrhosis, liver failure, and portal hypertension and often requires liver transplantation.

Liver cirrhosis is a chronic disease of the liver characterized by destruction of hepatocytes and vessels by inflammatory processes and collagen depositions. With continuous destruction of hepatocytes, the liver becomes shrunken and distorted in shape, forming multiple hepatocellular nodules separated by broad fibrotic bands, which disrupts intrahepatic blood circulation and induces portal hypertension with extensive portocarval shunts. The main complications of cirrhosis, such as gastroesophageal varices, ascites, hepatic encephalopathy, and renal and cardiac disorders. Hepatic fibrosis as well as liver cirrhosis are not pathognomonic of any particular disease. They can be the result of a variety of chronic disease states of the liver, e.g., chronic viral hepatitis, fatty liver (alcoholic, nonalcoholic form), chronic toxin exposure, liver congestion, chronic congestive hepatitis (e.g., in right heart failure), prolonged cholestasis, α1-antitrypsin deficiency.

Hepatocellular carcinoma is a malignant neoplasm that develops directly from hepatocytes. Usually, hepatocellular carcinoma is preceded by chronic liver cell damage, which includes cirrhosis, chronic infection with the hepatitis B virus, hepatitis C virus, excessive alcohol consumption, non-alcoholic fatty liver disease, obesity, type II diabetes mellitus, and smoking.

Hypercholesterolemia refers to a fat metabolism disorder (dyslipidemia). This is a condition characterized by elevated blood cholesterol levels. It includes primary, congenital cholesterolemia and secondary, acquired cholesterolemia. It is considered a significant risk factor for atherosclerosis and thus for the occurrence of numerous cardiovascular diseases. Causes of hypercholesterolemia can i.a. be obesity, diabetes, chronic renal insufficiency, or also hypothyroidism.

Insulin resistance is the decreased ability of cells to respond to the action of insulin in transporting glucose from the bloodstream to muscle, liver, and other tissues. Insulin resistance often develops in obesity and is associated with prediabetes and the onset of type 2 diabetes. Clinically, insulin resistance can be defined as the inability of a known amount of exogenous or endogenous insulin to increase an individual's glucose uptake and utilization as much as in a healthy patient.

Hyperglycemia is a condition in which there is an excessive concentration of glucose in the blood. Hyperglycemia can lead to neuropathy, vascular damage, and developmental disorders (25). Late effects include kidney damage, cardiovascular damage, retinal damage, or damage to the feet and legs. There can also occur an increased susceptibility to infections. Hyperglycemia can be the consequence of diabetes, the use of certain medications, a critical illness such as stroke or myocardial infarction, stress, or hormonal imbalances.

Diabetes is a disease that results in high blood glucose levels due to the body's inability to synthesize insulin in sufficient amounts. Diabetes includes: Type I diabetes, Type 2 diabetes, gestational diabetes, monogenic diabetes, i.a. diabetes mellitus in newborns, children and/or or adolescents, and diabetes associated with cystic fibrosis. Symptoms include increased thirst and urination, headache, fatigue, and blurred vision.

Metabolic syndrome is a group of conditions that includes hypertension, hyperglycemia, excess body fat, and abnormal blood cholesterol and/or triglyceride levels. It leads to an increased risk of cardiovascular disease, stroke and diabetes. Symptomatology can be similar to a diabetes patient. Insulin resistance is often associated with metabolic syndrome.

These diseases can occur in both humans and animals.

No active compounds have yet been described for use in the treatment of lipid metabolism disorders that are inhibitors that act mARC1 protein, resp. that act at the protein level to downregulate the activity of mARC1. This problem is solved with the compounds presented here, which surprisingly inhibit mARC1. The compounds of the invention, which are inhibitors of human mARC1, have the general structure I:

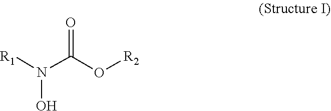

in which the residues R₁ and R₂ can be identical or different and are hydrogen (only in the case of R₁) and/or alkyl and/or aryl and/or heterocycles, in each case saturated or unsaturated and substituted and/or unsubstituted. The residues R₁ and R₂, in each case identical or different, can be alkyl, aryl or an aromatic residue, optionally with heteroatoms, e.g. C1- to C6-alkyl, linear, cyclic or branched, e.g. methyl, ethyl, propyl, butyl, pentyl, hexyl, C1- to C6-aryl or C6-aromatic, in each case optionally substituted, in particular substituted with at least one heteroatom or functional group, preferably phenyl, in each case optionally substituted, in particular in the meta position or in the 3-position, with at least one heteroatom which is preferably a halogen, e.g. fluorine, chlorine, bromine or iodine, e.g. chlorophenyl, preferably 3-chlorophenyl. In particular, R₁ and R₂ can each form 4-,5-,6-,7-, or 8-membered ring systems, each of which can be saturated or unsaturated as well as substituted and/or unsubstituted. R₁ and R₂ can comprise the following functional groups directly and/or as substitutions of the structural features mentioned: carboxylic acids, peroxycarboxylic acids, thiocarboxylic acids, sulfonic acids, sulfenic acids, sulfinic acids, sulfoxides, carboxylic acid anhydrides, carboxylic acid esters, sulfonic acid esters, carboxylic acid amides, sulfonic acid amides, carboxylic acid hydrazides, carboxylic acid halides, sulfonic acid halides, nitriles, aldehydes, ketones, thioaldehydes, thioketones, oximes, N-oxides, hydrazones, alcohols, phenols, thiols, amines, amidines, guanidines, imines, hydrazines, ethers, esters, thioethers, nitro groups, nitroso groups, azo groups, diazo groups, isocyanides, isocyanates, thiocyanates, isothiocyanates, hydroperoxides and/or peroxides . Optionally, R1 is an aromatic residue which can be substituted, or a C1- to C6- alkyl or aryl, in particular 3-chlorophenyl, and R2 is hydrogen, methyl, ethyl, propyl, pentyl or hexyl, in each case optionally substituted with at least one heteroatom, preferably ethyl. When R1 is hydrogen, R2 is preferably an aromatic residue which can be substituted with at least one heteroatom, or a C1 to C6 alkyl or aryl, especially methyl, ethyl, propyl, pentyl or hexyl, each optionally substituted with at least one heteroatom, preferably ethyl. The active compounds may contain only saturated C—C bonds or may contain at least one C—C double bond and/or C—C triple bond. The active compounds can be present as salts or as bases or acids. Likewise, the active compounds can be prodrugs or can be administered as prodrugs. In the case of chiral atoms, the invention relates to all enantiomers and diastereomers as well as all polymorphic forms. All prodrugs can be prepared by conventional methods or can be purchased from manufacturers. In general, it is preferred that at least one active compound according to the invention is contained in a pharmaceutical formulation for oral or systemic administration.

Preferred embodiments of the active compounds of structure I

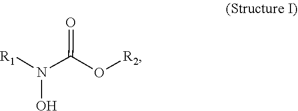

wherein, for example, R1 is hydrogen, an aromatic residue or a substituted aromatic residue, or a C1- to C6- alkyl group, and R2 is hydrogen, an aromatic residue or a substituted aromatic residue, or a C1- to C6- alkyl group, wherein only one of R1 and R2 can be hydrogen,
in particular

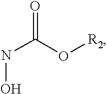

wherein R1 is hydrogen and R2 is an aromatic residue which can be substituted or a C1- to C6-alkyl, in particular methyl, ethyl, propyl, pentyl or hexyl, or aryl or an aromatic, preferably phenyl, in each case optionally substituted, in particular in the meta position or in the 3-position, with at least one heteroatom which is preferably a halogen, e.g. fluorine, chlorine, bromine or iodine, in particular 3-chlorophenyl,

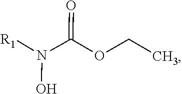

wherein R2 is ethyl and R1 is an aromatic residue which can be substituted, preferably phenyl, in each case optionally substituted, in particular in the meta position or in the 3-position, with at least one heteroatom which is preferably a halogen, e.g. fluorine, chlorine, bromine or iodine, in particular 3-chlorophenyl, or a C1- to C6-alkyl or aryl, in each case optionally substituted with at least one heteroatom,

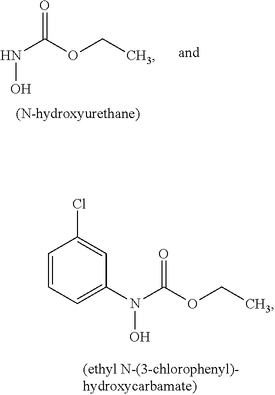

wherein R1 is hydrogen and R2 is ethyl,

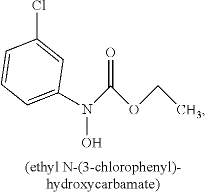

wherein R1 is 3-chlorophenyl and R2 is ethyl.

Inhibitors and retardands, which are active compounds, according to the invention mean all chemical compounds that lower or downregulate the conversion rate or activity of mARC1. Therein, downregulation in the patient means that a lower conversion rate is achieved in the patient by the inhibitors in reactions catalyzed by mARC1. Similarly, in the patient, protein-protein interactions involving mARC1 are downregulated by a medicament with the described compounds. Inhibitors also include all substances that inhibit the degradation of another medicament by mARC1 so that this is present in sufficiently high therapeutic concentration. This follows the principle of the pharmacokinetic booster and is analogous to the therapeutic combination of benserazide or resp. carbidopa with levodopa or to the combination of ritonavir with lopinavir. According to the invention, thereby inter alia the inactivation by mARC1 of functional groups that are elemental to the efficacy of another medicament, such as hydroxamic acids, amidoximes or hydroxylamines is prevented. Thus, medicaments such as antibiotics that possess such a functional group can be used in humans, wherein the active compound of the invention reduces the inactivation of the other medicament by inhibiting mARC1. Medicament means all pharmaceutical preparations comprising the inhibitors of the invention that together with excipients and carriers form a formulation, which are made available to a human orally, topically, rectally, subcutaneously, nasally. parenterally/intravenously, inhalatively or by other means. Accordingly, the invention also relates to active compounds that are inhibitors of mARC1 for use as a component of a pharmaceutical containing another pharmaceutical compound, e.g., containing, in addition to the inhibitor, another pharmaceutical compound comprising at least one hydroxamic acid, an amidoxime, and/or a hydroxylamine or consisting thereof.

Pharmaceuticals or pharmaceutical preparations containing an active compound according to the invention that reduces mARC1 expression or mARC1 activity, and optionally still another pharmaceutical compound comprising or consisting of at least one hydroxamic acid, an amidoxime and/or a hydroxylamine, can be prepared by any method known in pharmacy, e.g. by combining the active compound with the carrier(s) or excipient(s). A “pharmaceutical excipient,” “carrier,” or “pharmaceutically acceptable carrier,” as used herein, includes any solvent, dispersion medium, coating, antibacterial and antifungal compound, dye, isotonic and absorption retardant compound, and the like that is physiologically acceptable. Examples of pharmaceutically acceptable excipients are water, saline, phosphate-buffered saline, dextrose, glycerin, ethanol, and the like, and combinations thereof. In many cases, it can be beneficial to incorporate isotonic compounds, e.g., sugars, polyalcohols, e.g., mannitol, sorbitol, or sodium chloride, into the formulation. Pharmaceutically acceptable carriers can also contain small amounts of excipients such as wetting compounds, emulsifiers, preservatives, or buffers that improve the shelf life or efficacy of the active compound. In certain aspects, the active compound can be made with a carrier that protects the compound from rapid release, such as a controlled release formulation, including implants, transdermal patches, and microencapsulated delivery systems. Biodegradable, biocompatible polymers can be used in delivery systems, such as ethylene vinyl acetate, polyanhydrides, polyglycolic acid, collagen, polyorthoester, and polylactic acid. Many methods for preparing such drugs and formulations are known to pharmaceutical professionals.

Active compound-containing formulations can be present in different forms. The preferred form depends on the intended route of administration and therapeutic use, which in turn dictates the type of carrier/excipient. Suitable forms i.a. include liquid, semi-solid and solid dosage forms.

Pharmaceutical formulations adapted for oral administration can be administered, for example, as discrete units such as capsules or tablets, powders or granules, solutions or suspensions in aqueous or non-aqueous liquids, edible foams, or liquid oil-in-water emulsions or liquid water-in-oil emulsions. The active compound can be included in a formulation suitable for oral administration, e.g., by combining that active compound with an inert solvent or by incorporating it into an edible carrier compound. The active compound (and other ingredients, if desired) can also be enclosed in a hard or soft shell gelatin capsule, compressed into tablets, or incorporated directly into the subject's food. For oral therapeutic administration, the drugs can be mixed with excipients and used in the form of ingestible tablets, buccal tablets, lozenges, capsules, elixirs, suspensions, syrups, wafers and the like. To administer a compound of the invention by means other than parenteral administration, it can be necessary to coat the compound with a material or to co-administer the compound with a material to prevent its inactivation.

Pharmaceutical formulations designed for transdermal administration can e.g. be administered as patches suitable to remain in close contact with the epidermis of the recipient for a prolonged period of time, or as an electrode for iontophoretic administration.

Pharmaceutical formulations adapted for topical administration can be formulated, for example, as ointments, creams, suspensions, lotions, powders, solutions, pastes, gels, sprays, aerosols, or oils.

Pharmaceutical drugs or formulations suitable for nasal administration in which the carrier is a solid comprise a powder having a defined particle size or particle size range, for example in the range of 20 to 500 μm, which is administered in a manner analogous to the ingestion of snuff, i.e. by rapid inhalation through the nasal passage from a container of the powder held close to the nose. Suitable formulations in which the carrier is a liquid, which can be used as a nasal spray or as nasal drops, include aqueous or oily solutions of the active compound.

Pharmaceutical formulations suitable for administration by inhalation inter alia include finely divided dusts or mists, which can be produced by various types of metered dose pressurized inhalers, powder inhalers, nebulizers or atomizers (membrane nebulizers or nozzle nebulizers). In connection with the administration of the active compounds described herein by inhalation, inhalation medicaments, such as metered dose inhalers, as are commonly known in pharmaceutical applications, are used. Metered dose inhalers are configured to deliver a single dose of an active compound per actuation, although multiple actuations can be required to effectively treat a particular patient.

Pharmaceutical drugs or formulations suitable for parenteral administration include aqueous and non-aqueous sterile injection solutions that can contain, for example, antioxidants, buffers, bacteriostats, lipids, liposomes, emulsifiers, but also suspending compounds and modifiers for rheology. The formulations can be prepared in single-dose or multiple-dose containers, e.g., sealed ampoules and vials, and can be stored in a freeze-dried (lyophilized) state, requiring only the addition of the sterile liquid carrier, e.g., water for injections, immediately prior to use. Injection solutions and suspensions for immediate use can be prepared from sterile powders, granules and tablets.

Therapeutic pharmaceutics resp. formulations generally must be sterile and stable under the conditions of manufacture and storage. For example, sterile injectable solutions can be prepared by incorporating the active compound in the required amount into a suitable solvent containing one, or a combination, of the ingredients listed above, as required, and then sterile filtering. Generally, dispersions are prepared by incorporating the active compound into a sterile vehicle containing a simple dispersion medium and the required other ingredients from the ingredients listed above. In the case of sterile powders for the preparation of sterile injectable solutions, typical manufacturing processes include vacuum drying and freeze drying, in which a powder of the active compound and any other desired ingredient is obtained from a previously sterile filtered solution of the active compound. Proper flowability can be maintained, for example, by using a coating such as lecithin, by maintaining the required particle size in the case of a dispersion, and by using surfactants. A prolonged absorption of injectable pharmaceutical forms can be achieved by including into the composition a compound that retards absorption, e.g., monostearate salts and gelatin.

A “therapeutically effective amount” refers to an amount of a pharmaceutical compound or active compound that is effective at the dosages applied for the periods of time necessary to achieve the desired therapeutic outcome. A “therapeutically effective amount” for treating a condition is an amount of an active compound or of a dosage form, such as a single dose or multiple doses, that is effective to achieve an identifiable endpoint. The “effective amount” is preferably safe—at least to the extent in which the benefits of the treatment outweigh the harms and/or the harms are acceptable to a normal expert and/or to an appropriate regulatory authority, such as the European Medicines Agency (EMA). The therapeutically effective amount of an active compound can vary depending on factors such as disease state, age, sex, and weight of the individual, and the ability of the active compound to elicit a desired response in the individual. A “prophylactically effective amount” refers to an amount that is effective at the doses and for the periods of time necessary to achieve a desired prophylactic result. Because a prophylactic dose is used in individuals prior to or at an earlier stage of disease, the prophylactically effective amount can typically be lower than the therapeutically effective amount.

Dosing regimens can be adjusted to achieve the optimal desired response (e.g., a therapeutic or prophylactic response). For example, a single bolus can be administered, multiple individual doses can be administered over time, or the composition can be administered continuously or pulsed, with doses or partial doses being administered at regular intervals, e.g., every 10, 15, 20, 30, 45, 60, 90, or 120 minutes, every 2 to 12 hours, daily, or every other day, etc. Depending on the requirements of the therapeutic situation, the dose can be proportionally reduced or increased. In some cases, it can be particularly advantageous to formulate compositions, such as e.g. parenteral or inhaled dosage form, in the form of dosage units in order to ease administration and to standardize dosing. The specification of dosage units is dictated by and is directly dependent on (a) the unique properties of the active compound and the particular therapeutic or prophylactic effect to be achieved, and (b) the specifics of the individual patient.

When reference is made to the ability of a compound to specifically inhibit mARC1, it is meant that the active compound binds to mARC1. This can be carried out in vitro or in vivo within an acceptable tolerance range that provides a sufficiently specific diagnostic or therapeutic effect according to the standards of a person skilled in the art, a medical professional, and/or a regulatory authority such as the European Medicines Agency (EMA). This happens in the context of inhibition of mARC1 and downregulation of mARC1 activity in the effective treatment of liver disease, non-alcoholic fatty liver disease, non-alcoholic steatohepatitis, liver cirrhosis, liver fibrosis, hepatocellular carcinoma. Decreased cholesterol levels, cardiovascular disease, insulin resistance, impaired glucose tolerance, high glucose levels, type II diabetes mellitus, metabolic syndrome, and/or a condition resulting from any of the foregoing in a patient as described herein.

In another embodiment, the active compound is coupled to a binding reagent. A binding reagent is a reagent, compound, or composition, such as e.g. a ligand that is capable of specifically binding a target compound, such as mARC1. A binding reagent can interfere with mARC1 activity, e.g., as an inhibitor or antagonist of mARC1. Binding reagents are e.g. small molecules, i.a. antibodies (polyclonal, monoclonal, preferably humanized), antibody fragments (e.g., a recombinant scFv), antibody mimetics, engineered proteins, antigens, epitopes, haptens, or any binding reagent specific for mARC1. As a class, binding reagents include: small molecules, monoclonal antibodies or derivatives or analogs thereof, including without limitation: Fv fragments, single chain Fv (scFv) fragments, Fab′ fragments, F(ab′)2 fragments, single domain antibodies and antibody fragments, humanized antibodies and antibody fragments, multivalent versions of the foregoing, and any paratope-containing compounds or compositions; multivalent activators, including and without exception: monospecific or bispecific antibodies, such as e.g. disulfide-stabilized Fv fragments, scFv tandems ((scFv)2 fragments), nucleic acids, and analogs thereof that bind a target compound; or receptor molecules that naturally interact with a desired target molecule. Anti-mARC1 antibodies are commercially available or can be prepared by a person skilled in the art using standard methods.

The term “inhibit” as most commonly used herein is interchangeable with, “retard”, “downregulate,” “suppress,” and other similar terms and includes any degree of inhibition.

The figures show:

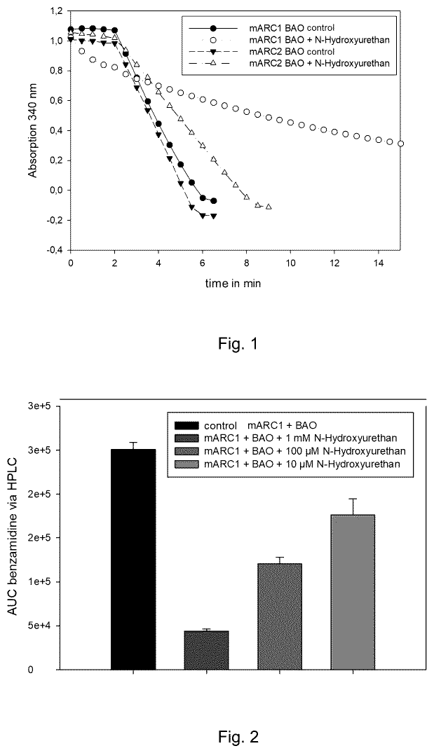

FIG. 1: The reaction rates of mARC1 and mARC2 measured with and without the addition of N-hydroxyurethane (1 mM). Benzamidoxime (BAO) was added as substrate after 2 minutes. The experiment was terminated when no more NADH was present (no change in absorbance at 340 nm) or after 15 minutes had elapsed.

FIG. 2: Overview of the benzamidine (reduced benzamidoxime) recovery using different concentrations of N-hydroxyurethane. The results are shown as AUC of an HPLC analysis in duplicate.

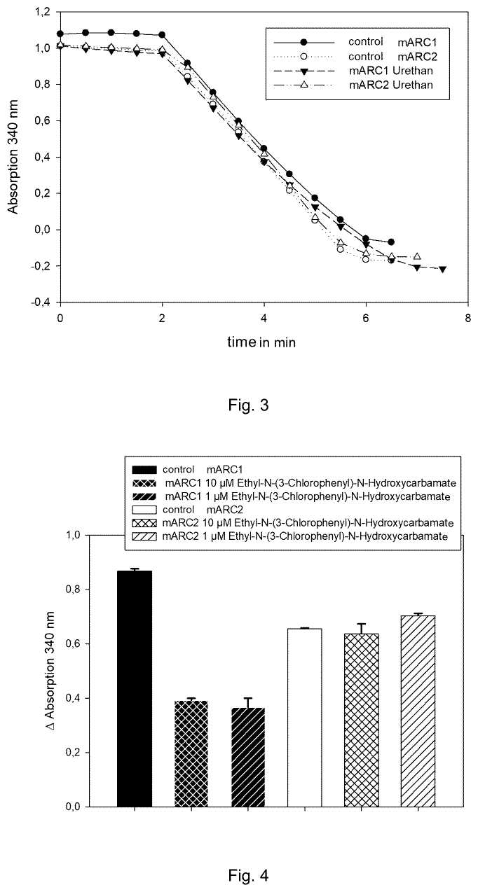

FIG. 3: Reaction rates of mARC1 and mARC2 with and without the addition of urethane (1 mM). Benzamidoxime was added as substrate after 2 minutes. The experiment was terminated when no more NADH was present (no change in absorbance at 340 nm) or after 15 minutes had elapsed.

FIG. 4: Comparison of the change in absorbance at 340 nm over 10 minutes of different incubation batches with and without ethyl-N-(3-chlorophenyl)-N-hydroxycarbamate for mARC1 and mARC2.

### EXAMPLES: N-HYDROXYURETHANE OR ETHYL-N-(3-CHLOROPHENYL)-HYDROXYCARBAMATE AS ACTIVE COMPOUND TO INHIBIT mARC1

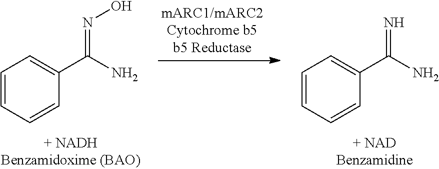 [SMILES: N/C(=N\O)c1ccccc1 ; N=C(N)c1ccccc1]

The compounds N-hydroxyurethane (R₁═H, R₂═CH₂CH₃) and ethyl-N-(3-chlorophenyl) -hydroxycarbamate (R₁═3-chlorophenyl, R₂═CH₂CH₃) are used as examples of active compounds. FIGS. 1, 3 and 4 show the absorbance at 340 nm in dependence on the time for an incubation with the three-component system mARC1 and mARC2, respectively, cytochrome b5 and cytochrome b5 reductase, which catalyze the conversion of the model substrate benzamidoxime (BAO) to benzamidine in the presence of the cosubstrate NADH (5). The decrease in the concentration of NADH due to its absorption maximum at 340 nm is thereby measured. From the measurement results, which are shown in the bar graph of FIG. 4, the inhibition of the conversion of benzamidoxime to benzamidine is evident by the lower consumption of NADH. NADH represents the required cosubstrate for the conversion. FIG. 2 shows the concentration of benzamidine, the product of the conversion, measured by HPLC. The inhibition occurs only in the presence of active compounds according to structure I, here represented by N-hydroxyurethane (FIGS. 1, 2) or by the substituted ethyl-N-(3-chlorophenyl)-hydroxycarbamate (FIG. 4), not with urethane itself (FIG. 3, see structure I, but hydrogen instead of the OH group). The down-regulation is only observed for mARC1 and not for mARC2 (FIG. 2). From the non-inhibited conversion for mARC2, it can also be seen that cytochrome b5 and/or cytochrome b5 reductase are not inhibited.

### A Currently Assumed Reaction Mechanism Is

N-hydroxyurethane, as an inhibitor of mARC1, can bind to its serine 271 in the active site. This is located at a distance of 7.6 Å from the molybdenum cofactor of mARC1. Therein, the N—O bond of N-hydroxyurethane is coordinated at the molybdenum cofactor's molybdenum ion, as postulated for mARC. Thereby, the carbonyl function from the carbamate is brought into direct vicinity to the hydroxy function of the serine 271 (comprehensible from the mARC1 crystal structure (16, 17)). This leads to carbamylation of the serine with cleavage of ethanol. The active site of mARC1 is thereby blocked. The carbamylated serine can return to the original state by a reaction with water. This reaction is mARC1-specific, since mARC2 in this position has a proline in place of serine.

### General Instructions for Experiments (acc. to 5)

105 μl MES buffer (20 mM, pH 6.0) was placed in a micro-UV cuvette. To this, 20 μl each of mARC (7.5 μg per batch), CYB5B (75 pmol per batch) and CYB5R3 (7.5 pmol per batch) were added. The amount of CYB5B and CYB5R3 used refers to the respective cofactor. In those control experiments where not all enzymes were present, the missing volume was adjusted with MES buffer instead of the respective enzyme. 60 μl of inhibitor was then pipetted to this mixture. The concentration of inhibitor in the final vessel varied depending on the experimental requirement. The total batch consisting of buffer, protein and inhibitor was pre-incubated for 3 min, at 37° C.in the heating block of the photometer. This batch was then set as a blank sample and the absorbance was zeroed. 60 μl of NADH (1 mM) was then added to this incubation batch and the measurement was started immediately. Herein, the absorbance spectrum between 300 nm and 400 nm was recorded every 30 s for a period of 10 min (spectrophotometer Cary® 50 Bio with Walter peltier system PCB 150, Agilent). After starting the reaction with NADH, 15 μl BAO (60 mM) was added after 2 min. The reaction proceeded for another 8 min or was stopped if 3 consecutive readings at 340 nm showed no changes. The control measurement was performed analogously for an incubation without inhibitor. The conversion rates of BAO with and without inhibitor were compared based on the absorbance change at 340 nm to determine the degree of inhibition. The calculation was made using the molar extinction coefficient of NADH and follows the principle that the consumption of NADH is equimolar to the conversion of the substrate benzamidoxime (BAO). The validation is described in Indorf et al. (5). The HPLC analysis was performed according to the methodology described by Ginsel et al (3).

### Recovery of the Recombinant Proteins

Protein sources: The expression and purification of human mARC1 (reference sequence NP_073583) and human mARC2 (reference sequence NP_060368), CYB5B (reference sequence NP_085056), and CYB5R3 (reference sequence NP_000389) was performed in Escherichia coli as described by Wahl and colleagues (50). The protein content was determined using the BCA protein assay kit (Pierce, Rockford, IL, USA) according to the manufacturer's protocol. The heme content in CYB5B was determined by recording the difference spectrum of oxidized and NADH-reduced protein (51). The FAD content in CYB5R3 was measured according to Whitby at 450 nm (52). The sample was obtained after heating at 100° C.for 10 min and centrifugating at 22000×g for 5 min at room temperature. A calibration curve (0.01 mM to 0.1 mM) was used for quantification of FAD.

### Reagents

| Reagent | Manufacturer |
|---|---|
| Benzamidoxime | Synthesis according to (4) |
| Ethyl-N-(3-chlorophenyl)-N- | SIGMA-ALDRICH CHEMIE GmbH, |
| hydroxycarbamate | Steinheim, Germany |
| 2-(N-morpholino)ethanesulfonic | Carl Roth GmbH + Co. KG, Karlsruhe |
| acid (MES) | Merck KGaA, Darmstadt |
| NADH disodium salt | Tokyo Chemical Industry, Eschborn |
| N-hydroxyurethane | SIGMA-ALDRICH CHEMIE GmbH, |
| Urethane | Steinheim, Germany |

### Bibliography

1. Havemeyer A et al. (2006) Identification of the missing component in the mitochondrial benzamidoxime prodrug-converting system as a novel molybdenum enzyme. The Journal of biological chemistry 281:34796-34802.
2. Ott G, Havemeyer A, Clement B (2015) The mammalian molybdenum enzymes of mARC. Journal of biological inorganic chemistry : JBIC : a publication of the Society of Biological Inorganic Chemistry 20:265-275.
3. Ginsel C et al. (2018) The Involvement of the Mitochondrial Amidoxime Reducing Component (mARC) in the Reductive Metabolism of Hydroxamic Acids. Drug metabolism and disposition: the biological fate of chemicals 46:1396-1402.
4. Jakobs HH et al. (2014) The mitochondrial amidoxime reducing component (mARC): involvement in metabolic reduction of N-oxides, oximes and N-hydroxyamidinohydrazones. ChemMedChem 9:2381-2387.
5. Indorf P, Kubitza C, Scheidig AJ, Kunze T, Clement B (2019) Drug Metabolism by the Mitochondrial Amidoxime Reducing Component (mARC): Rapid Assay and Identification of New Substrates. Journal of medicinal chemistry.
6. Sparacino-Watkins CE et al. (2014) Nitrite reductase and nitric-oxide synthase activity of the mitochondrial molybdopterin enzymes mARC1 and mARC2. The Journal of biological chemistry 289:10345-10358.
7. Kotthaus J et al. (2011) Reduction of N(ω)-hydroxy-L-arginine by the mitochondrial amidoxime reducing component (mARC). The Biochemical journal 433:383-391.
8. Neve EPA et al. (2012) Amidoxime reductase system containing cytochrome b5 type B (CYB5B) and MOSC2 is of importance for lipid synthesis in adipocyte mitochondria. The Journal of biological chemistry 287:6307-6317.
9. Jakobs HH et al. (2014) The N-reductive system composed of mitochondrial amidoxime reducing component (mARC), cytochrome b5 (CYB5B) and cytochrome b5 reductase (CYB5R) is regulated by fasting and high fat diet in mice. PloS one 9:e105371.
10. Krompholz N et al. (2012) The mitochondrial Amidoxime Reducing Component (mARC) is involved in detoxification of N-hydroxylated base analogues. Chemical research in toxicology 25:2443-2450.
11. Emdin CA et al. (2020) A missense variant in Mitochondrial Amidoxime Reducing Component 1 gene and protection against liver disease. PLOS Genet 16:e1008629.
12. Malik AN, Rossios C, Al-Kafaji G, Shah A, Page RA (2007) Glucose regulation of CDK7, a putative thiol related gene, in experimental diabetic nephropathy. Biochemical and biophysical research communications 357:237-244.
13. Chen X et al. (2010) Novel Association Strategy with Copy Number Variation for Identifying New Risk Loci of Human Diseases. PloS one 5:e12185.
14. Rixen S et al. (2019) Mitochondrial amidoxime-reducing component 2 (MARC2) has a significant role in N -reductive activity and energy metabolism. The Journal of biological chemistry 294:17593-17602.
15. Altschul SF, Gish W (1996) in Computer methods for macromolecular sequence analysis, ed Doolittle RF. (Acad. Press, San Diego, Calif.), pp 460-480.
16. Xu J et al. (2008) Fibroblast Growth Factor 21 Reverses Hepatic Steatosis, Increases Energy Expenditure, and Improves Insulin Sensitivity in Diet-Induced Obese Mice. Diabetes 58:250-259.
17. Younossi ZM et al. (2016) Global epidemiology of nonalcoholic fatty liver disease-Meta-analytic assessment of prevalence, incidence, and outcomes. Hepatology 64:73-84.
18. Dai Wet al. (2017) Prevalence of nonalcoholic fatty liver disease in patients with type 2 diabetes mellitus. Medicine 96:e8179.
19. Machado M, Marques-Vidal P, Cortez-Pinto H (2006) Hepatic histology in obese patients undergoing bariatric surgery. Journal of Hepatology 45:600-606.
20. Estes C, Razavi H, Loomba R, Younossi Z, Sanyal AJ (2018) Modeling the epidemic of nonalcoholic fatty liver disease demonstrates an exponential increase in burden of disease. Hepatology 67:123-133.
21. Zhang X-J, She Z-G, Li H (2018) Time to step-up the fight against NAFLD. Hepatology 67:2068-2071.
22. Younossi ZM et al. (2016) The economic and clinical burden of nonalcoholic fatty liver disease in the United States and Europe. Hepatology 64:1577-1586.
23. Perumpail BJ et al. (2017) Clinical epidemiology and disease burden of nonalcoholic fatty liver disease. WJG 23:8263-8276.
24. Wong RJ, Cheung R, Ahmed A (2014) Nonalcoholic steatohepatitis is the most rapidly growing indication for liver transplantation in patients with hepatocellular carcinoma in the U.S. Hepatology 59:2188-2195.
25. Mikolasevic I et al. (2018) Nonalcoholic fatty liver disease and liver transplantation—Where do we stand? WJG 24:1491-1506.
26. Shimada M et al. (2002) Hepatocellular carcinoma in patients with non-alcoholic steatohepatitis. Journal of Hepatology 37:154-160.
27. Singal AG et al. (2014) The Effect of PNPLA3 on Fibrosis Progression and Development of Hepatocellular Carcinoma. A Meta-analysis. American Journal of Gastroenterology 109:325-334.
28. Matteoni C et al. (1999) Nonalcoholic fatty liver disease. A spectrum of clinical and pathological severity⋆, ⋆⋆. Gastroenterology 116:1413-1419.
29. Michelotti GA, Machado MV, Diehl AM (2013) NAFLD, NASH and liver cancer. Nat Rev Gastroenterol Hepatol 10:656-665.
30. El-Serag HB, Rudolph KL (2007) Hepatocellular Carcinoma. Epidemiology and Molecular Carcinogenesis. Gastroenterology 132:2557-2576.
31. Lallukka S, Yki-Järvinen H (2016) Non-alcoholic fatty liver disease and risk of type 2 diabetes. Best Practice & Research Clinical Endocrinology & Metabolism 30:385-395.
32. Targher G, Day CP, Bonora E (2010) Risk of Cardiovascular Disease in Patients with Nonalcoholic Fatty Liver Disease. N Engl J Med 363:1341-1350.
33. Savage DB, Petersen KF, Shulman GI (2007) Disordered Lipid Metabolism and the Pathogenesis of Insulin Resistance. Physiological Reviews 87:507-520.
34. Savage DB (2006) Reversal of diet-induced hepatic steatosis and hepatic insulin resistance by antisense oligonucleotide inhibitors of acetyl-CoA carboxylases 1 and 2. Journal of Clinical Investigation 116:817-824.
35. Samuel VT, Shulman GI (2016) The pathogenesis of insulin resistance. Integrating signaling pathways and substrate flux. Journal of Clinical Investigation 126:12-22.
36. Sachithanandan N et al. (2010) Liver-specific suppressor of cytokine signaling-3 deletion in mice enhances hepatic insulin sensitivity and lipogenesis resulting in fatty liver and obesityl. Hepatology 52:1632-1642.
37. Biddinger SB et al. (2008) Hepatic Insulin Resistance Is Sufficient to Produce Dyslipidemia and Susceptibility to Atherosclerosis. Cell Metabolism 7:125-134.
38. (2015) Diabetes Mellitus Predicts Occurrence of Cirrhosis and Hepatocellular Cancer in Alcoholic Liver and Non-alcoholic Fatty Liver Diseases. JCTH 3:9-16.
39. Min H-K et al. (2012) Increased Hepatic Synthesis and Dysregulation of Cholesterol Metabolism Is Associated with the Severity of Nonalcoholic Fatty Liver Disease. Cell Metabolism 15:665-674.
40. Ismaiel A, Dumitraşcu DL (2019) Cardiovascular Risk in Fatty Liver Disease. The Liver-Heart Axis-Literature Review. Front. Med. 6:e28.
41. Go AS et al. (2014) Heart Disease and Stroke Statistics—2014 Update. Circulation 129:1.
42. Marchesini G (2003) Nonalcoholic fatty liver, steatohepatitis, and the metabolic syndrome. Hepatology 37:917-923.
43. Kucukazman M et al. (2013) Evaluation of early atherosclerosis markers in patients with nonalcoholic fatty liver disease. European Journal of Gastroenterology & Hepatology 25:147-151.
44. Teli MR, Day CP, James OFW, Burt AD, Bennett MK (1995) Determinants of progression to cirrhosis or fibrosis in pure alcoholic fatty liver. The Lancet 346:987-990.
45. Stickel F, Hoehn B, Schuppan D, Seitz HK (2003) Nutritional therapy in alcoholic liver disease. Aliment Pharmacol Ther 18:357-373.
46. Liu J-D et al. (1998) Alcohol-Related Problems in Taiwan with Particular Emphasis on Alcoholic Liver Diseases. Alcoholism: Clinical and Experimental Research 22:164S-169S.
47. Nascimbeni F et al. (2013) From NAFLD in clinical practice to answers from guidelines. Journal of Hepatology 59:859-871.
48. Larson-Meyer DE et al. (2008) Effect of 6-Month Calorie Restriction and Exercise on Serum and Liver Lipids and Markers of Liver Function. Obesity 16:1355-1362.
49. Keating SE et al. (2015) Effect of aerobic exercise training dose on liver fat and visceral adiposity. Journal of Hepatology 63:174-182.
50. Wahl B et al. (2010) Biochemical and spectroscopic characterization of the human mitochondrial amidoxime reducing components hmARC-1 and hmARC-2 suggests the existence of a new molybdenum enzyme family in eukaryotes. The Journal of biological chemistry 285:37847-37859.
51. Estabrook RW, Werringloer J (1978) The measurement of difference spectra. Application to the cytochromes of microsomes. Methods in enzymology 52:212-220.
52. Whitby LG (1953) A new method for preparing flavin-adenine dinucleotide. The Biochemical journal 54:437-442.

1. Active compound for use in the prevention and treatment of lipid metabolism disorders, in particular obesity, non-alcoholic fatty liver diseases, alcoholic fatty liver diseases, non-alcoholic steatohepatites, liver cirrhoses, liver fibroses, elevated liver enzyme levels (ALT, AST, ALP), hepatocellular carcinomas, hypercholesterolemiae and associated cardiovascular diseases, insulin resistance, impaired glucose tolerance, hyperglycemia, type II diabetes mellitus, and metabolic syndrome, wherein the active compound is an inhibitor of mARC1.

2. Active compound for use in the prevention and treatment of lipid metabolism disorders according to claim 1, wherein the active compound has structure I: 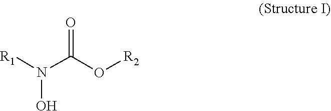
in which the residues R1 and R2 are identical or different and are selected from hydrogen and/or C1- to C6-alkyl, linear, cyclic or branched and/or C1- to C6-aryl, C6-aromatics and/or heterocycles, each substituted by at least one heteroatom or functional group or unsubstituted, and 4-, 5-, 6-, 7- or 8-membered ring systems.

3. Active compound for use in the prevention and treatment of lipid metabolism disorders according to claim 2, wherein the functional group is selected from carboxylic acid groups, peroxycarboxylic acid groups, thiocarboxylic acid groups, sulfonic acid groups, sulfenic acid groups, sulfinic acid groups, sulfoxide groups, carboxylic acid anhydride groups, carboxylic acid ester groups, sulfonic acid ester groups, carboxylic acid amide groups, sulfonic acid amide groups, carboxylic acid hydrazide groups, carboxylic acid halide groups, sulfonic acid halide groups, nitrile groups, aldehyde groups, ketone groups, thioaldehyde groups, thioketone groups, oxime groups, N-oxide groups, hydrazone groups, alcohol groups, phenol groups, thiol groups, amine groups, amidine groups, guanidine groups, imine groups, hydrazine groups, ether groups, ester groups, thioether groups, nitro groups, nitroso groups, azo groups, diazo groups, isocyanide groups, isoscyanate groups, thiocyanate groups, isothiocyanate groups, hydroperoxide groups, and peroxide groups.

4. Active compound for use in the prevention and treatment of lipid metabolism disorders according to claim 2, wherein R1 is hydrogen and R2 is an aromatic residue which can be substituted or a C1- to C6-alkyl or aryl, in each case optionally substituted with at least one heteroatom, preferably phenyl, in each case optionally substituted, in particular substituted in the meta-position or in the 3-position with at least one heteroatom which is preferably a halogen, e.g. fluorine, chlorine, bromine or iodine, in particular 3-chlorophenyl.

5. Active compound for use in the prevention and treatment of lipid metabolism disorders according to claim 2, wherein R2 is ethyl and R1 is an aromatic residue, optionally substituted, in particular 3-chlorophenyl, or a C1- to C6-alkyl or C6-aryl, optionally substituted.

6. Active compound for use in the prevention and treatment of lipid metabolism disorders according to claim 2, characterized in that the active compound has the structure I wherein R1 is hydrogen and R2 is ethyl, or the structure I where R1 is 3-chlorophenyl and R2 is ethyl.

7. Active compound for use in the prevention and treatment of lipid metabolism disorders according to claim 1, wherein the active compound is contained in a pharmaceutic which, in addition to the inhibitor of mARC1, also contains another pharmaceutical compound comprising or consisting of at least one hydroxamic acid, an amidoxime and/or a hydroxylamine.

8. Active compound for use in the prevention and treatment of lipid metabolism disorders according to claim 1, wherein the active compound induces the downregulation of reactions catalyzed by mARC1 and of protein-protein interactions mediated by mARC1.

9. Active compound for use in the prevention and treatment of lipid metabolism disorders according to claim 1, wherein the active compound induces the lowering of lipids in the blood of the patient who has received the inhibitor.


SEQUENCE LISTING (2 sequences)

SEQ ID NO: 1 - RNA, 2258 nt, Homo sapiens
  Homo sapiens mitochondrial amidoxime reducing component 1 (MARC1), mRNA
acagcgcccugcagcgcaggcgacggaagguugcagaggcaguggggcgccgaccaaguggaagcugagccaccaccucccacuccccgcgccgccccccagaaggacgcacugcucugauuggcccggaaggguucaggagcugcccagccuuugggcucggggccaaaggccgcaccuucccccagcggccccgggcgaccagcgcgcuccggccuugccgccgccaccucgcggagaagccagccaugggcgccgccggcuccuccgcgcuggcgcgcuuuguccuccucgcgcaaucccggcccggguggcucgggguugccgcgcugggccugaccgcgguggcgcugggggcugucgccuggcgccgcgcauggcccacgcggcgccggcggcugcugcagcaggugggcacaguggcgcagcucuggaucuacccugugaaauccugcaagggggugccggugagcgaggcggagugcacggccauggggcugcgcagcggcaaccugcgggacagguuuuggcuugugaucaaccaggagggaaacaugguuacugcucgccaggaaccucgccugguccugauuucccugaccugcgauggugacacccugacucucagugcagccuacacaaaggaccuacuacugccuaucaaaacgcccaccacaaaugcagugcacaagugcagagugcacggccuggagauagagggcagggacuguggcgaggccaccgcccaguggauaaccagcuuccugaagucacagcccuaccgccuggugcacuucgagccucacaugcgaccgagacguccucaucaaauagcagacuuguuccgacccaaggaccagauugcuuacucagacaccagcccauucuugauccuuucugaggcgucgcuggcggaucucaacuccaggcuagagaagaaaguuaaagcaaccaacuucaggcccaauauuguaauuucaggaugcgaugucuaugcagaggauucuugggaugagcuucuuauuggugacguggaacugaaaagggugauggcuuguuccagaugcauuuuaaccacaguggacccagacaccggugucaugagcaggaaggaaccgcuggaaacacugaagaguuaucgccagugugacccuucagaacgaaaguuauauggaaaaucaccacucuuugggcaguauuuugugcuggaaaacccagggaccaucaaagugggagacccuguguaccugcugggccaguaaugggaaccguauguccuggaauauuagaugccuuuuaaaaauguucucaaaaaugacaacacuugaagcaugguguuucagaacugagaccucuacauuuucuuuaaauuugugauuuucacauuuuucgucuuuuggacuucuggugucucaaugcuucaaugucccagugcaaaaaguaaagaaauauagucucaauaacuuaguaggacuucaguaagucacuuaaaugacaagacaggauucugaaaacuccccguuuaacugauuauggaauaguucuuucuccugcuucuccguuuaucuaccaagagcgcagacuugcauccugucacuaccacucguuagagaaagagaagaagagaaagaggaagagugggugggcuggaagaauauccuagaauguguuauugccccuguucaugagguacgcaaugaaaauuaaauugcaccccaaauauggcuggaaugccacuucccuuuucuucucaagccccgggcuagcuuuugaaauggcauaaagacugaggugaccuucaggaagcacugcagauauuaauuuuccauagaucuggaucuggcccugcugcuucucagacagcauuggauuuccuaaaggugcucaggaggaugguuguguagucauggaggaccccuggauccuugccauuccccucagcuaaugacggagugcuccuucuccaguuccgggugaaaaaguucugaauucuguggaggagaagaaaagugauucagugauuucagauagacuacugaaaaccuuuaaagggggaaaaggaaagcauaugucaguuguuuaaaacccaauaucuauuuuuuaacugauuguauaacucuaagaucugaugaaguauauuuuuuauugccauuuuguccuuugauuauauugggaaguugacuaaacuugaaaaauguuuuuaaaacugugaauaaauggaagcuacuuugacuaguuucagaaaaaaaaaaaaaaaa

SEQ ID NO: 2 - protein, 337 aa, Homo sapiens
  MARC1_HUMAN Mitochondrial amidoxime-reducing component 1 Homo sapiens
MGAAGSSALARFVLLAQSRPGWLGVAALGLTAVALGAVAWRRAWPTRRRRLLQQVGTVAQLWIYPVKSCKGVPVSEAECTAMGLRSGNLRDRFWLVINQEGNMVTARQEPRLVLISLTCDGDTLTLSAAYTKDLLLPIKTPTTNAVHKCRVHGLEIEGRDCGEATAQWITSFLKSQPYRLVHFEPHMRPRRPHQIADLFRPKDQIAYSDTSPFLILSEASLADLNSRLEKKVKATNFRPNIVISGCDVYAEDSWDELLIGDVELKRVMACSRCILTTVDPDTGVMSRKEPLETLKSYRQCDPSERKLYGKSPLFGQYFVLENPGTIKVGDPVYLLGQ